(19) United States
(12) Patent Application Publication
(10) Pub. No.: US 2026/0035668 A1
(43) Pub. Date: Feb. 5, 2026

(54) ORGANOID CULTURE COMPOSITION FOR INDUCING RESERVE STEM CELLS AND CULTURE METHOD USING SAME

(71) Applicant: KOREA UNIVERSITY RESEARCH AND BUSINESS FOUNDATION, Seoul (KR)

(72) Inventors: Yong Taek JEONG, Seodaemun-gu (KR); Dong Woo SEO, Dongdaemun-gu (KR)

(73) Assignee: KOREA UNIVERSITY RESEARCH AND BUSINESS FOUNDATION, Seoul (KR)

(21) Appl. No.: 18/852,181

(22) PCT Filed: Mar. 29, 2023
(86) PCT No.: PCT/KR2023/004180

§ 371 Date: Apr. 16, 2025

(30) Foreign Application Priority Data
Mar. 29, 2022 (KR) 10-2022-0038697

(51) Int. Cl. C12N 5/071

(52) U.S. Cl. CPC C12N 5/0679; C12N 2501/11; C12N 2506/45; C12N 2509/10; C12N 2513/00

(57) ABSTRACT

The present invention relates to: a cell or organoid culture composition for inducing reserve stem cells comprising SAG as an active ingredient; a culture additive composition; a medium comprising the composition; a method for preparing a reserve stem cell-containing organoid; a reserve cell-containing organoid prepared by the preparation method; and a method for culturing the organoid. The reserve stem cell-containing organoid can be formed by culturing an organoid with the culture composition comprising SAG as an active ingredient of the present invention, since the reserve stem cell-containing organoid has resistance against various harmful stimuli, such as radiation exposure, and can be safely transplanted and engrafted to a patient scheduled for radiation exposure, etc., the reserve stem cell-containing organoid can be used in various ways in regenerative medicine.

### TECHNICAL FIELD

The present disclosure relates to a cell or organoid culture composition for inducing reserve stem cells, comprising SAG as an active ingredient, a culture additive composition, a medium comprising the culture additive composition, a method for preparing a reserve stem cell-containing organoid; a reserve stem cell-containing organoid prepared by the preparation method, and a method for culturing the organoid.

### BACKGROUND ART

Organoids are in vitro organ mimics prepared by three-dimensional culture of cells isolated from stem cells or organ-derived cells, and are expected to be available for various diseases because organoid fabrication technology theoretically allows almost any type of organ to be prepared with only stem cells. Organoids may be more effective than two-dimensionally grown cell tissues in testing safety and efficacy of new drugs, and may be utilized to improve conditions by transplanting them in damaged or underdeveloped organs. Accordingly, research into organoids has recently become more active in terms of regenerative medicine, and it is expected that organoids will be widely used in various fields.

It is most important that the transplanted organoids survive for a long period of time and engraft in recipient's tissues in utilizing organoids in regenerative medicine. In order to apply organoids to patients with tissue and organ defects caused by various harmful stimuli, it is necessary for the organoids to be transplanted to have resistance to harmful stimuli.

Meanwhile, there are two types of stem cells: adult stem cells that have an activated cell cycle, and reserve stem cells that usually maintain a resting phase of the cell cycle and may act as adult stem cells when adult stem cells are deficient due to harmful stimuli that cause DNA damage. Reserve stem cells are resistant to external damage, and thus organoids differentiated into reserve stem cells may have improved resistance to various stimuli and environmental factors.

Organoids formed through conventional organoid culture techniques contain significantly fewer reserve stem cells, although they contain adult stem cells, and thus have lower resistance to various harmful stimuli such as radiation exposure. Therefore, there is a high possibility of failure when such organoids are transplanted and engrafted in patients scheduled for radiation exposure.

Under these circumstances, the inventors of the present disclosure have made great efforts to culture organoids enriched in reserve stem cells, and have confirmed that reserve stem cells were formed when the organoids were cultured with a medium composition according to the present disclosure comprising SAG, thereby completing the present disclosure.

### DISCLOSURE

### Technical Problem

An object of the present disclosure is to solve the foregoing problems and other problems related thereto.

One exemplary object of the present disclosure is to provide a cell or organoid culture composition for inducing reserve stem cells, comprising smoothened receptor agonist (SAG) as an active ingredient.

Another exemplary object of the present disclosure is to provide a medium for culturing cells or organoids for inducing reserve stem cells, comprising the cell or organoid culture composition as described above.

Another exemplary object of the present disclosure is to provide a cell or organoid culture additive composition for inducing reserve stem cells, comprising SAG as an active ingredient.

Another exemplary object of the present disclosure is to provide a method for preparing a reserve stem cell-containing organoid, comprising culturing an adult stem cell-derived organoid in the cell or organoid culture composition as described above.

Another exemplary object of the present disclosure is to provide a reserve stem cell-containing organoid prepared by the method as described above.

Another exemplary object of the present disclosure is to provide a method for culturing a reserve stem cell-containing organoid, comprising culturing an adult stem cell-derived organoid in the cell or organoid culture composition as described above.

Another exemplary object of the present disclosure is to provide use of the reserve stem cell-containing organoid as described abve, for transplantation or engraftment into an individual receiving radiation therapy.

The technical problems to be achieved according to the technical idea of the present disclosure disclosed in the present specification are not limited to the problems for solving the above-mentioned problems, and other problems that are not mentioned may be obviously understood by those skilled in the art from the following description.

### Technical Solution

This will be specifically described as follows. Meanwhile, each of the descriptions and embodiments disclosed in the present application may also be applied to each other descriptions and embodiments. That is, all combinations of various elements disclosed in the present application fall within the scope of the present application. In addition, the scope of the present application cannot be considered limited by the specific description set forth below.

One aspect of the present disclosure to achieve the above object provides a cell or organoid culture composition for inducing reserve stem cells, comprising smoothened receptor agonist (SAG) as an active ingredient.

As used herein, the term “stem cells” refers to undifferentiated cells that are capable of differentiating into various cells constituting biological tissues, and are capable of regenerating itself in an unrestricted manner to form specialized cells of tissues and organs. The term “adult stem cells” refers to stem cells that appear at a stage where each organ of embryo is formed as a development process progresses or at an adult stage.

The adult stem cells may be largely divided into activated adult stem cells and reserve stem cells. The reserve stem cells are also called quiescent stem cell, and unlike activated adult stem cells that have an activated cell cycle, they are usually maintain a resting phase of the cell and may act as adult stem cells when activated stem cells are deficient due to various harmful stimuli that cause DNA damage and are resistant to external damage.

As used herein, the term “culture” means proliferating, growing, maintaining, and differentiating cells isolated from a living organism, a two-dimensional or three-dimensional aggregate thereof, a tissue, or a part of the tissue in vitro. Thus, the “culture” refers to encompassing an entire process of obtaining a target substance under an artificial environment using a starting material (cell, tissue, or organoid, which is a tissue analog).

As used herein, the term “smoothened receptor agonist (SAG)” refers to a compound represented by the following Formula 1, which is a key part of the hedgehog signaling pathway and is known to prevent brain damage caused by drugs.

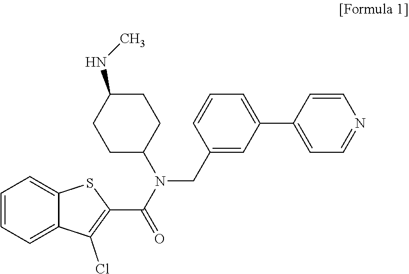

In the present disclosure, the culture composition may comprise 0.1 to 2 μM of SAG, specifically 0.5 to 1.5 μM of SAG, and more specifically 1 μM of SAG, but the present disclosure is not limited thereto.

As used herein, the term “organoid” refers to an in vitro 3D cellular cluster comprising primary tissue, tissue subunits, or single cells (e.g., stem cells). Organoids are capable of self-renewal and self-organization, and reproduce phenotypes and functions similar to those of the original tissue, and may therefore be called miniature pseudo-organs or organ analogues.

In order to utilize the organoids in regenerative medicine, the organoids transplanted into the human body need to survive for a long period of time and engraft in the recipient's tissues. Therefore, in order to apply organoids to patients with tissue and organ defects caused by various harmful stimuli, it is necessary for the organoids to be transplanted to have properties that resist harmful stimuli such as radiation. In this regard, the foregoing reserve stem cells are resistant to external damage, such that the reserve stem cell-containing organoid may have improved resistance to various stimuli and environmental factors.

In the present disclosure, the organoid cultured with the composition may contain reserve stem cells and thus has radiation resistance.

In the present disclosure, the organoid cultured with the composition may have a higher expression level of a TERT protein or a gene encoding the TERT protein than before culture.

In the present disclosure, the organoid may be derived from small intestine or large intestine.

Specifically, in an embodiment of the present disclosure, organoids were inoculated and cultured by supplementing advanced DMEM F/12 medium with a HEPES buffer, glutamax, N-acetylcysteine, N2, B-27, RSPO (R-spodin), noggin, EGF, and Y-27632, followed by incubation with CHIR99021 and valproic acid, and then treated with SAG and re-cultured to prepare a reserve stem cell-containing organoid. It was confirmed that the reserve stem cell-containing organoid had resistance to radiation and expressed a cell proliferation marker (Ki-76) at a significantly higher level than a control group.

In the present disclosure, the culture composition may further comprise one or more agent selected from CHIR99021 or a pharmaceutically acceptable salt thereof, and valproic acid or a pharmaceutically acceptable salt thereof.

According to a specific embodiment of the present disclosure, the culture composition may comprise 1 to 10 μM of CHIR99021, specifically 2 to 8 μM of CHIR99021, and more specifically 3 μM of CHIR99021.

According to a specific embodiment of the present disclosure, the culture composition may comprise 0.1 to 2 mM of valproic acid, specifically 0.5 to 1.5 mM of valproic acid, and more specifically 1 mM of valproic acid.

In the present disclosure, the composition may further comprise one or more selected from HEPES, glutamax, N-acetylcysteine, N2, B-27, R-spodin, noggin, EGF, and Y-27632.

In the present disclosure, the culture composition may comprise a basal medium, wherein the basal medium may be one selected from the group consisting of Dulbecco's Modified Eagle's Medium (DMEM), Minimal Essential Medium (MEM), Basal Medium Eagle (BME), RPMI 1640, F-10, F-12, DMEM/F12, advanced DMEM/F12, α-Minimal Essential Medium (α-MEM), Glasgow's Minimal Essential Medium (G-MEM), Iscove's Modified Dulbecco's Medium (IMDM), MacCoy's 5A medium, AmnioMax, AmnioMax complete Medium, Chang's Medium, MesenCult-XF Medium, and specifically, advanced DMEM/F12.

Another aspect of the present disclosure for achieving the above object provides a medium for culturing cells or organoids for inducing reserve stem cells, comprising the culture composition.

The reserve stem cells, organoids, and culture are as described above.

As used herein, the term “medium” refers to a medium capable of supporting proliferation, survival, and differentiation of organoids, and includes all conventional media suitable for the culture and differentiation of organoids used in the art. The type of medium and the culture conditions may be appropriately selected depending on the type of organoid.

Another aspect of the present disclosure for achieving the above object provides a cell or organoid culture additive composition for inducing reserve stem cells, comprising SAG as an active ingredient.

The SAG, reserve stem cells, organoids, and culture are as described above.

As used herein, the term “culture additive” refers to a component of a culture medium, and in one embodiment, it may be added in large quantities to a culture medium in a conventional state to provide SAG to the culture medium of the present disclosure at a required concentration according to the present disclosure. In addition, it is also possible to prepare the culture medium of the present disclosure by adding various components to the culture additive of the present disclosure to form the culture medium of the present disclosure.

In the present disclosure, the culture additive composition may further comprise one or more selected from CHIR99021 or a pharmaceutically acceptable salt thereof, and valproic acid or a pharmaceutically acceptable salt thereof.

In the present disclosure, the culture additive composition may further comprise one or more selected from HEPES, glutamax, N-acetylcysteine, N2, B-27, R-spodin, noggin, EGF, and Y-27632.

Another aspect of the present disclosure for achieving the above object provides a method for producing a reserve stem cell-containing organoid, comprising the step of culturing an adult stem cell-derived organoid in the culture composition.

The reserve stem cells, organoids, and culture are as described above.

In the present disclosure, the adult stem cells may be derived from small intestine or large intestine, and specifically, may be derived from the small intestine, but the present disclosure is not limited thereto.

Another aspect of the present disclosure for achieving the above object provides a reserve stem cell-containing organoid prepared by the preparation method.

The reserve stem cells and organoids are as described above.

Another aspect of the present disclosure for achieving the above object provides a method for culturing a reserve stem cell-containing organoid, comprising culturing an adult stem cell-derived organoid in the culture composition.

The reserve stem cells, organoids, and culture are as described above.

The culture method may include the steps of:

(a) inoculating organoids into advanced DMEM/F12 medium;

(b) performing culture by adding CHIR99021 and valproic acid to the medium inoculated with the organoids; and

(c) performing culture by treating the culture cultured in step (b) with SAG.

In the present disclosure, the advanced DMEM/F12 medium of step (a) may further comprise one or more selected from HEPES, glutamax, N-acetylcysteine, N2, B-27, R-spodin, noggin, EGF, and Y-27632.

According to a specific embodiment of the present disclosure, the concentration of the SAG may be 0.1 to 2 μM, specifically 0.5 to 1.5 μM, and more specifically 1 μM, but the present disclosure is not limited thereto.

According to a specific embodiment of the present disclosure, the concentration of the CHIR99021 may be 1 to 10 μM, specifically 2 to 8 μM, and more specifically 3 μM.

According to a specific embodiment of the present disclosure, the concentration of the valproic acid may be 0.1 to 2 mM, specifically 0.5 to 1.5 mM, and more specifically 1 mM.

Another aspect of the present disclosure for achieving the above object provides use of the reserve stem cell-containing organoid, for transplantation or engraftment into an individual receiving radiation therapy.

In the present disclosure, the individual receiving radiation therapy refers to all individuals before, during, or after receiving radiation therapy.

Another aspect of the present disclosure provides a composition for transplanting or engrafting into an individual receiving radiation therapy, comprising the reserve stem cell-containing organoid, or a method for transplanting or engrafting the reserve stem cell-containing organoid into an individual receiving radiation therapy.

In the present disclosure, the individual includes, but is not particularly limited to, for example, human, monkey, cow, horse, sheep, pig, chicken, turkey, quail, cat, dog, mouse, rat, rabbit, or guinea pig, and preferably means mammal, and more preferably human.

### Advantageous Effects

The reserve stem cell-containing organoid can be formed by culturing an organoid with the culture composition comprising SAG according to the present disclosure as an active ingredient, where since the reserve stem cell-containing organoid has resistance to various harmful stimuli, such as radiation exposure, and can be safely transplanted and engrafted to a patient scheduled for radiation exposure, etc., the reserve stem cell-containing organoid can be used in various ways in regenerative medicine.

However, the effects according to one embodiment of the technology disclosed in this specification are not limited to those mentioned above, and other effects that are not mentioned will be obviously understood by those skilled in the art from the following description.

### DESCRIPTION OF DRAWINGS

A brief description of each drawing is provided to more fully understand the drawings cited herein.

FIG. 1 illustrates the results of confirming the activity of a reserve stem cell marker TERT of a reserve stem cell-containing organoid according to the present disclosure.

FIG. 2 illustrates the results of confirming radiation resistance of a reserve stem cell-containing organoid according to the present disclosure.

FIG. 3 illustrates the results of confirming a radiation resistance through the activity of a cell proliferation marker Ki-67 of a reserve stem cell-containing organoid according to the present disclosure.

FIG. 4 illustrates the results of confirming a differentiation function of a reserve stem cell-containing organoid according to the present disclosure.

### BEST MODE

Hereinafter, the present disclosure will be described in more detail with reference to the following Examples. However, these Examples are intended to illustrate the present disclosure by way of example, and the scope of the present disclosure is not limited to these Examples.

### EXAMPLE 1

### Preparation of Reserve Stem Cell-Containing Organoid

### 1-1. Preparation of Organoids

To prepare small intestine-derived organoids, small intestine tissue was isolated from 4 to 8 week-old C57Bl/6 mice and perfused with PBS. The tissue was cut into pieces of 2 to 4 mm in size, placed in PBS, and washed clearly about 10 times. Then, the tissue was placed in PBS containing 200 mM EDTA, and incubated at 4° C. for 1 hour with shaking. Thereafter, the cells detached from the tissue were passed through a 70 um cell strainer, and centrifuged at 200 g. Organoids were prepared by mixing Matrigel in a 1:1 volume ratio with a culture medium containing settled small intestinal crypts and then performing three-dimensional culture.

### 1-2. Culture for Induction of Reserve Stem Cell Differentiation

The medium was prepared by adding a 10 mM HEPES buffer, 1X glutamax, 1 mM N-acetylcysteine, 1 volume of N2, 2 volumes of B-27, 20 volumes of RSPO (R-spodin), 10 volumes of noggin per 100 volumes of total medium, and 50 ng/ml of EGF to advanced DMEM F/12 medium (Gibco™, Cat. No 12634010) as a basic medium for small intestine organoids.

Subsequently, 10 μM Y-27632 was added to the medium, and the organoids prepared in Example 1-1 were inoculated and cultured for 3 days. Then, 3 μM CHIR99021 (Sigma-Aldrich, Cat. No SML1046-5 MG) and 1 mM valproic acid (Sigma-Aldrich, Cat. No P4543-10G) were added and incubated for 4 days at 37° C. in a 5% CO2 incubator, followed by treatment with 1 μM SAG (Abcam, Cat. No ab142160) and incubation to culture the reserve stem cell-induced organoids.

As a control group, organoids were cultured except for the treatment of SAG in the above process.

### EXPERIMENTAL EXAMPLE 1

### Verification of Differentiated Reserve Stem Cells in Organoids

It was confirmed whether reserve stem cells were formed in the cultured reserve stem cell-induced organoids cultured in Example 1.

Specifically, the organoids were fixed with 4% PFA, and 0.1% Triton X-100 in PBS was used to increase a permeability of the organoids. Subsequently, 3% BSA and 0.1% Triton X-100 were placed in a solution diluted in PBS for 1 hour to block a membrane of the organoids, and incubated with primary antibodies (anti-hTERT (ab32020, abcam), anti-Ki67 (ab16667, abcam), anti-Green Fluorescent Protein (GFP-1020, aveslabs), anti-Dclk1 (ab31704, abcam), anti-UEA 1 (RL-1062-2, Vector Laboratories)) at 4° C. for 1 day. After washing with PBS three times for 10 minutes at room temperature, incubation was performed with fluorescent secondary antibodies corresponding to each primary antibody for 2 hours. After washing gain with PBS three times for 10 minutes at room temperature, the organoid nuclei were stained with 4′, 6-diamidino-2-phenylindole, dihydrochloride (DAPI) for 5 minutes at room temperature.

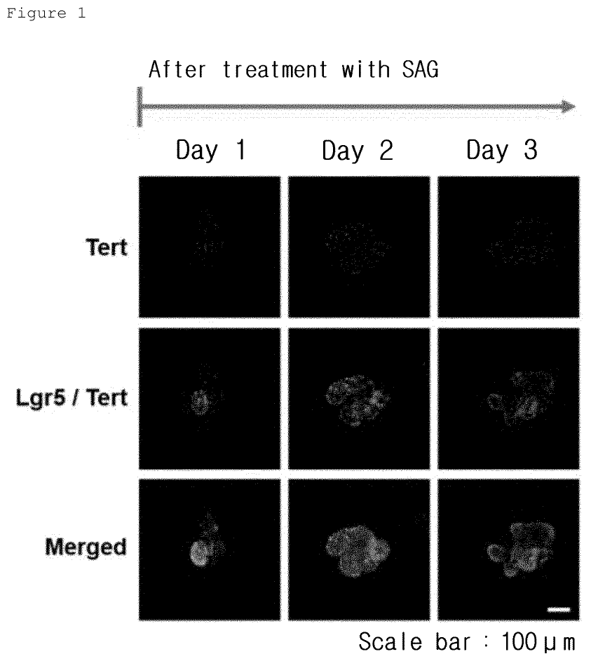

The results showed that an activity of TERT significantly increased over time after incubation of organoids by treatment with SAG, confirming that SAG is involved in the differentiation of reverse cells (FIG. 1).

### EXPERIMENTAL EXAMPLE 2

### Analysis of Radiation Resistance of Reserve Stem Cell-Containing Organoids

In order to confirm the radiation resistance of reserve stem cell-derived organoids prepared in Example 1, the formation of small intestinal crypts and the expression of a cell proliferation marker Ki-67 were confirmed after radiation expose.

Specifically, 3 μM CHIR99021 and 1 mM Valproic acid were added to advanced DMEM F/12 medium supplemented with HEPES, glutamax, N-acetylcysteine, N2, B-27, R-spodin, noggin, and EGF. Thereafter, the organoids prepared in Example 1-1 were inoculated and cultured for 6 days, and then cultured for 3 days each under the conditions shown in the table below.

TABLE 1

| Number | Culture conditions | classification |
|---|---|---|
| \textcircled{1} | Advanced DMEM F/12 medium with | Control group 1 |
| | HEPES, glutamax, N- | |
| | acetylcysteine, N2, B-27, | |
| | R-spodin, noggin, and EGF | |
| \textcircled{2} | \textcircled{1} + 3 μM CHIR99021 and 1 mM | Control group 2 |
| | Valproic acid | |
| \textcircled{3} | \textcircled{2} + 1 μM SAG | Experimental |
| | | group |

Then, the organoids cultured under each culture condition were irradiated once with 6 Gy of X-ray using a radiation irradiator (rs-320), and the formation of small intestinal crypts was confirmed after 72 hours.

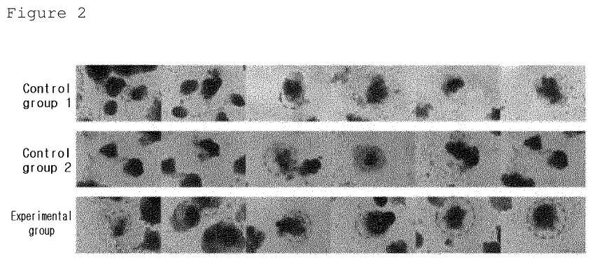

As a result, the experimental group treated with SAG showed significantly better formation of small intestinal crypts compared to the other control groups, indicating significantly higher radiation resistance than the other control groups (FIG. 2). Thus, it can be seen that the reverse stem cell-induced organoids cultured with the culture medium of the present disclosure comprising SAG have a protective effect on the reduction of cell death due to radiation exposure during organ transplantation.

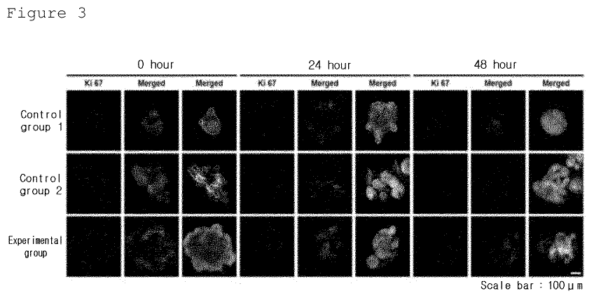

In addition, at 0 hour, 24 hours, and 48 hours after the completion of the culture of the organoid cultured under each culture condition, a first antibody to an antigenic determinant of a target protein was attached in the same manner as in Experimental Example 1, and then a fluorescent secondary antibody capable of binding to the first antibody was attached. The expression of the cell proliferation marker Ki-67 was confirmed by immunofluorescence to confirm the expression level of the target protein. As a result, it was confirmed that in the experimental group to which the culture medium of the present disclosure comprising SAG was applied, Ki-67 was expressed at a significantly higher level than a control group at each time point (FIG. 3).

### EXPERIMENTAL EXAMPLE 3

### Verification of Differentiation Potential of Reserve Stem Cell-Containing Organoids

It was confirmed whether the reserve stem cell-containing organoids cultured in Example 1 could be differentiated into secretory cells.

Specifically, small intestinal organoids were cultured in the same manner as in Example 1-2, and small intestinal stem cell markers Dclkl (tuft cells) and UEA-1 (goblet cells) were verified using immunofluorescence. As a control group, the small intestinal organoids cultured using only advanced DMEM F/12 medium supplemented with HEPES, glutamax, N-acetylcysteine, N2, B-27, R-spodin, noggin, and EGF were used.

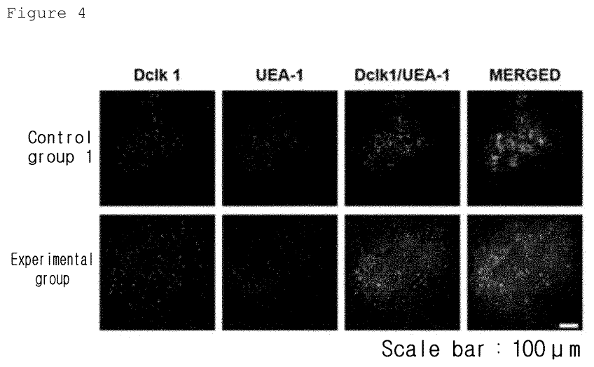

As a result, as shown in FIG. 4, when cultured with the culture medium of the present disclosure comprising SAG (experimental group), Dclk1 and UAE-1 were significantly increased compared to the control group not comprising SAG, confirming that the reserve cells in the organoid cultured with the culture composition of the present disclosure can normally differentiate into various mature cells.

From the above description, those skilled in the art to which the present disclosure pertains will understand that the present disclosure can be implemented in other specific forms without changing its technical idea or essential features. In this regard, it should be understood that the embodiments described above are exemplary in all respects and are not intended to be limiting. The scope of the present disclosure should be interpreted that all changes or modifications derived from the meaning and scope of patent claims to be described below rather than the detailed description above and their equivalent concepts are included in the scope of the present disclosure.

1. A cell or organoid culture composition for inducing reserve stem cells, comprising smoothened receptor agonist (SAG) as an active ingredient.

2. The cell or organoid culture composition of claim 1, further comprising one or more selected from CHIR99021 or a pharmaceutically acceptable salt thereof, and valproic acid or a pharmaceutically acceptable salt thereof.

3. The cell or organoid culture composition of claim 1, further comprising one or more selected from HEPES, glutamax, N-acetylcysteine, N2, B-27, R-spodin, noggin, EGF, and Y-27632.

4. The cell or organoid culture composition of claim 1, wherein the organoid cultured with the composition has radiation resistance.

5. The cell or organoid culture composition of claim 1, wherein the organoid cultured with the composition has a higher expression level of a TERT protein or a gene encoding the TERT protein than before culture.

6. The cell or organoid culture composition of claim 1, wherein the composition comprises a basal media.

7. The cell or organoid culture composition of claim 6, wherein the basal media is one selected from the group consisting of Dulbecco's Modified Eagle's Medium (DMEM), Minimal Essential Medium (MEM), Basal Medium Eagle (BME), RPMI 1640, F-10, F-12, DMEM/F12, advanced DMEM/F12, α-Minimal Essential Medium (α-MEM), Glasgow's Minimal Essential Medium (G-MEM), Iscove's Modified Dulbecco's Medium (IMDM), MacCoy's 5A medium, AmnioMax, AmnioMax complete Medium, Chang's Medium, and MesenCult-XF Medium.

8. The cell or organoid culture composition of claim 1, wherein the organoid is derived from small intestine or large intestine.

9-12. (canceled)

13. A method for preparing a reserve stem cell-containing organoid, comprising culturing an adult stem cell-derived organoid in the cell or organoid culture composition of claim 1.

14. The method of claim 13, wherein the adult stem cell is derived from small intestine or large intestine.

15. The method of claim 13, wherein the reserve stem cell-containing organoid has radiation resistance.

16. A reserve stem cell-containing organoid prepared by the method of claim 13.

17. The reserve stem cell-containing organoid of claim 16, wherein the organoid is a small intestine organoid or a large intestine organoid.

18. The reserve stem cell-containing organoid of claim 16, wherein the organoid has radiation resistance.

19-23. (canceled)
